(19) United States
(12) Patent Application Publication
(10) Pub. No.: US 2017/0049537 A1
(43) Pub. Date: Feb. 23, 2017

(54) SURFACE-MODIFIED HYBRID SURFACE IMPLANT AND METHOD FOR MANUFACTURING THE SAME

(71) Applicant: Jung Tae LEE, Seoul (KR)

(72) Inventors: Jung Tae LEE, Seoul (KR); Se Jun PARK, Seoul (KR); Seung Hwan LEE, Seoul (KR)

(21) Appl. No.: 15/307,670

(22) PCT Filed: Apr. 30, 2014
(86) PCT No.: PCT/KR2014/003867

§ 371 Date: Oct. 28, 2016

(51) Int. Cl. A61C 8/00; A61L 27/54; A61L 27/32; A61C 13/00; A61L 27/30

(52) U.S. Cl. CPC A61C 8/0015; A61C 13/0016; A61L 27/306; A61L 27/32; A61L 27/54

(57) ABSTRACT

The present invention relates to an implant having a nanotube on a surface thereof and a method for manufacturing the same, and more particularly, to an implant having excellent stability and effectiveness because a nanotube is provided on the surface of the implant and a nanocapsule loaded with a medicine or the like is attached to the nanotube, thereby strengthening the adhesive force with the peripheral soft tissue and allowing to stably load factors related to osteogenesis and inflammation prevention and treatment or the like, thus not making them lost during an implant procedure and therefore not only having an increased lifespan but also preventing almost all occurrences of side effects caused by the implant.

### TECHNICAL FIELD

The present invention relates to a surface-modified hybrid surface implant and a method for manufacturing the same, more particularly, to a method for manufacturing an implant with an overall micro- and nano-scaled hybrid surface while ensuring the homogeneity of existing rough surfaced implants by modifying existing micro-scaled, rough surfaced implants into a nano-scaled implant. Also, the present invention relates to an implant endowed with a DDS function by virtue of the use of nanocapsules used for surface modification, and a method for manufacturing the same.

### BACKGROUND ART

Dental implant is a type of dental treatment in which a biocompatible implant body is inserted into the jawbone of the area where tooth is missing or extracted, with an additional surgery such as bone grafting, distraction osteogenesis, etc. In dental implant, after osseointegration or osteointegration, which is a morphological, physiological, direct connection between the normally functioning jawbone and the surface of the inserted implant body, is completed, the jawbone around the implant goes through bone remodeling. Dental implants can be classified as subperiosteal implant, endosseous implant, transosteal implant, etc., based on the site of insertion. They can also be classified as screw-type implant, cylinder-type implant, etc., based on their shape. Recently, the use of implant has proliferated, since it does not require drilling of an adjacent tooth and prevents alveolar bone resorption, resulting in functional and aesthetic excellence.

However, existing implants not only show an incomplete attachment between themselves and the soft tissue, but inevitably cause downward movement of the junctional epithelium and involve a gap in the attachment area, allowing easy bacterial penetration and thus leading to the problem of frequent occurrences of inflammation. In other words, these implants may cause gingivitis around themselves, and even have a reduced life span. In order to solve these problems, some implants are coated at the surface with a substance such as an antibiotic, an osteogenesis promoting factor, etc. before insertion. However, since torque is applied during the insertion, these have a problem that in the end, an antibiotic and an osteogenesis promoting factor are not left in the implant insertion site due to the physical force, and most of them are lost.

There is a technology in which a surface-engineered dental implant is coated with a recombinant osteogenesis promoting protein and implanted in the jawbone (Korean Patent Laid-Open No. 10-2007-0068240). In this technology, the surface of a dental implant is engineered, coated with recombinant promoting protein molecules, and lyophilized under negative pressure to manufacture an implant. However, in the case of an implant manufactured according to this method, it is highly likely that all the substances coated on the surface may be lost due to the physical force applied during the insertion procedure.

In addition, existing implants composed of titanium or titanium alloy have difficulty in achieving initial stability since they have poor initial adhesion to the bone tissue. In order to solve this problem, a method of coating the surface of an implant with hydroxyapatite (HAp) powders has been developed. However, in the case of simply coating hydroxyapatite (HAp) onto the surface of an implant, the hydroxyapatite (HAp) coating peels off from the surface of the implant in the process of inserting the implant into the jawbone, due to the friction between the implant surface and the jawbone, giving rise to a problem that there is a drastically decreased or very little effect resulting from hydroxyapatite (HAp).

Adhesion between an implant and the bone tissue may be strong, and in particular, the adhesion of titanium may be relatively strong. However, it is preferable to further enhance the adhesion. To this end, numerous studies have been conducted from various perspectives. Currently, there is a method of increasing the implant surface roughness, in which relatively large irregularities are formed on the implant surface in order to further enhance the implant-bone adhesion, and the increased surface roughness provides a larger contact and settling area, resulting in an enhanced mechanical binding force and strength.

As described above, the surface of existing (dental) implants was developed from a machined surface to a rough surface with irregularities. Later, this technology was further developed to maximize the roughness, leading to the development of an implant obtaining such a rough surface with a surface treatment such as RBM, SLA, laser treatment, etc. Currently available rough surfaced implants have a micro-scale roughness. Although their roughness was maximized, they have problems that they are non-directional in terms of homogeneity or the method for controlling the homogeneity is very limited. Also, an approach has been developed for attaching a functional factor to an implant surface. However, functional factors cannot properly function since, when implanted in vivo, the expression time thereof is very short or they decompose immediately.

In order to overcome the above drawbacks, it is necessary to modify the surface so that it is homogeneous while having a micro-scaled roughness. Also, there is a need to develop an implant having a sufficient stability and allowing the insertion while achieving a high adhesive force between the implant and the bone and the soft tissue, and the surface of which is capable of being stably loaded with a medicine such as an antibiotic, osteogenesis promoting factor, angiogenic factor, etc.

### SUMMARY OF INVENTION

### Technical Problem

The present invention is to manufacture an implant having an overall micro- and nano-scaled hybrid surface while ensuring the homogeneity of existing rough surfaced implants and also enabling the control of the degree thereof, by modifying existing micro-scaled rough surfaced implants into a nano-scaled implant. In addition, the present invention is to endow an implant with a DDS function by using nanocapsules used for surface modification.

### Solution to Problem

The present inventors have found that by modifying an implant surface only with the procedure of modifying an existing micro-scaled rough surfaced implant into a nano-scaled implant, it is possible to secure a certain degree of homogeneity in an existing rough surfaced implant, thus inducing more rapid settling down of cells involved in osteogenesis and thereby inducing rapid wound healing and regeneration of solid bony tissue. Thereby, the present inventors have completed the present invention.

### Advantageous Effects of Invention

The present invention has the advantage of inducing rapid settling down of cells involved in osteogenesis and thereby inducing rapid wound healing and regeneration of solid bony tissue, by modifying the surface of an existing rough surfaced implant which is non-directional in terms of homogeneity into a nano-scaled surface, giving a homogeneity to the existing non-directional rough surfaced implant and thereby changing the implant surface from hydrophobic one to hydrophilic one.

In addition, the present invention makes it possible to induce stable and rapid osteanagenesis by attaching nanocapsules such as hydroxyapatite (HA), tricalcium phosphate (TCP), TiO₂, etc., which act as an excellent scaffold in osteogenesis, on the surface. The roughness and homogeneity of the surface may be adjusted by a method such as sonication, etc.

Meanwhile, hollow nanocapsules may be prepared with various sizes and thicknesses (100 nm˜500 nm in size, 20 nm˜100 nm in thickness). First, the size of nanocapsules used as a template is determined by adjusting the size of silica particles used as the core of the nanoparticles. In the case of silica, silica nanoparticles with various sizes may be prepared by the Stober method. In the sol-gel reaction, the type of solvent, amount of catalyst, amount of water, and the amount of tetraethylorthosilicate (TEOS), which is a precursor, etc. affect the size of particles. First, a certain concentration of tetraethylorthosilicate (TEOS), which is a precursor, is dissolved in quantified anhydrous ethanol in a reaction vessel. Thereafter, water is added to tetraethylorthosilicate, resulting in removal of tetraethylether by hydrolysis and substitution thereinto of the hydroxyl group of water. During this reaction, addition of ammonia solution is carried out, since spherical nanoparticles are uniformly formed under the basic condition. In particular, with uniform dropwise addition of water through a separating funnel with stirring, the solution of tetraethylorthosilicate in anhydrous ethanol forms silica precursors slightly to prepare a silica nanoparticles dispersion. Meanwhile, core-shell silica particles with single mesopores are prepared as follows: Silica nanoparticles dispersion is added to distilled water including ammonia solution, etc. in a reaction vessel, and the resultant is put into a solvent in which distilled water mixed with cetyltrimethylammonium bromide (CTABr), 1,3,5-trimethylbenzene (TMB), and decane and ethanol are mixed at the ratio of 2:1 and then stirred. A silica template can be prepared by adding silica precursors with stirring and heating them. For the preparation of a composite of capsule type silica-hydroxyapatite, tricalcium phosphate (TCP), TiO₂, etc., the silica template prepared in the above step is put into a mixed solution of an organic solvent and water and dispersed. Precursor substances such as hydroxyapatite (HA), tricalcium phosphate (TCP), TiO₂, etc. are added to a mixed solution of an organic solvent such as ethylene glycol and water and then stirred to disperse the precursors uniformly into the solution. Here, when the precursor solution is added dropwise to the dispersed silica template, followed by vigorous stirring to proceed with reaction, coating starts with deposition of the precursor on the surface of the silica template through hydrolysis. The coating thickness is determined by the concentration and drop time of the precursor solution. After addition for a sufficient time of 30 minutes or more and stirring is completed, the coated capsule type composite of silica-hydroxy apatite (HA), tricalcium phosphate (TCP), TiO₂, etc. is centrifuged by using a centrifuge. Impurities adhered to these particles are washed with an organic solvent and air drying is performed. When thermal drying is carried out at 60° C. or higher, a pure capsule type silica composite from which solvent and impurities have been completely removed can be prepared. Thereafter, a porous hollow capsule is prepared as follows: The capsule type silica composite prepared is uniformly dispersed in a solvent in which water and an organic solvent is mixed, and silica and strong base are appropriately diluted in an aqueous solution, followed by heating and stirring for 30 minutes or more. In this process, the silica template in the core reacts with the base ion of the aqueous solution which penetrated thereinto to release silica gradually, resulting in the preparation of a hollow capsule from which the core has been removed. It is possible to change the properties of the capsule surface or raw materials. Also, since it is possible to load various functional factors thereon, it may serve as a drug delivery system, and enable to produce an implant allowing customized therapy.

### BRIEF DESCRIPTION OF DRAWINGS

FIG. 1 illustrates the development process of an implant surface.

FIG. 2 shows the shape of hollow nanocapsules of several properties.

FIG. 3 shows an electron micrograph of a cell which settled down onto the irregular shape of an implant surface.

FIG. 4 shows an electron micrograph of a cell which settled down onto the irregular shape of an implant surface.

FIG. 5 shows the experimental result indicating that when the surface is homogeneous, cells settle down rapidly.

FIG. 6 shows a photo of an irregular surface of an implant currently on the market.

FIG. 7 and FIG. 8 show a very irregular surface of implants currently on the market.

FIG. 9 shows a photo of SiO₂ templates.

FIG. 10 shows a photo of TiO₂ nanocapsules.

FIG. 11 shows a photo of the first hybrid surface implant according to the present invention.

FIG. 12 to FIG. 14 shows a photo of the second hybrid surface implant according to the present invention.

FIG. 15 shows a photo of SiO₂ beads.

### DESCRIPTION OF EMBODIMENTS

The present invention provides an overall micro- and nano-scaled hybrid surface implant while ensuring the homogeneity of existing rough surfaced implants and also enabling the control of the degree thereof, by modifying existing micro-scaled rough surfaced implants into a nano-scaled implant.

According to the present invention, it is possible to obtain both the advantage of existing rough surfaced implants and that of implants with a homogeneous surface. Also, since the present invention allows to manufacture an implant with a homogeneous surface only by carrying out the process of surface modification on an existing implant, it enables to conveniently manufacture an implant with a hybrid surface.

The present invention also provides an implant to which nanocapsules loaded with functional factors exhibiting a DDS function are attached.

### Embodiments

Hereinafter, the present invention will be described in more detail.

The present invention relates to a method for manufacturing an implant with a hybrid surface, comprising the steps of: (a) treating the surface of an implant fixture with sonication, and washing it with a solvent; (b1) preparing a SiO₂ bead, and preparing a nano-sized capsule by using it as a template, wherein a biocompatible material is coated on the SiO₂ template and then the SiO₂ bead is removed to prepare a hollow nanocapsule, or (b2) preparing a nanocapsule with a biocompatible material; and (c) attaching the hollow capsule prepared in step (b1) or the bead prepared in step (b2) to the implant fixture prepared in step (a) by dipping, stirring or centrifugation to manufacture a first hybrid surface implant.

In one embodiment according to the present invention, step (b1) may further include the step of loading a functional factor into the hollow capsule.

In one embodiment according to the present invention, an implant manufactured according to the above method has an overall uniform surface due to the nanocapsules attached to the surface.

In one embodiment according to the present invention, the biocompatible material in step (b1) or step (b2) may be selected from the group consisting of a titanium oxide selected from the group consisting of TiO₂, Ti₃O, Ti₂O, Ti₃O₂, TiO, Ti₂O₃, Ti₃O₅ and titanium butoxide; tricalcium phosphate, calcium phosphate; apatite selected from the group consisting of hydroxyapatite, hydroxyapatite substituted with silicon and magnesium; calcium sulfate; zirconium dioxide; silicon dioxide; and combinations thereof, and more specifically, selected from the group consisting of TiO₂, hydroxyapatite and tricalcium phosphate.

In one embodiment according to the present invention, in step (b1), the diameter of the SiO₂ nanocapsule may be not less than 500 nm and not more than 1 μm, more specifically, 500 nm, 600 nm, 700 nm, 800 nm, 900 nm, 1 μm.

In one embodiment according to the present invention, the functional factor may be selected from the group consisting of factors exhibiting the functions of promoting osteogenesis and enhancing antibacterial activity, anti-inflammatory activity and acidity, growth hormones, cell differentiation inducers, and angiogenic factors.

In one embodiment according to the present invention, the functional factor may be loaded into the hollow nanocapsule by using one or more method selected from the group consisting of dipping, centrifugation and sonication.

In another embodiment according to the present invention, an implant with a hybrid surface manufactured according to the above method is provided.

In one embodiment according to the present invention, the implant is characterized in that it is a dental implant.

The nanocapsule according to the present invention may be manufactured according to the following procedure:

### (1) Preparation of a Silica Nanoparticles Dispersion

Silica nanoparticles with various sizes can be prepared by using the Stober method. In the sol-gel reaction, the type of solvent, amount of catalyst, amount of water, and the amount of tetraethylorthosilicate (TEOS), which is a precursor, etc. affect the size of particles. Ethanol and water are put into a reaction vessel and then catalyst is added, followed by stirring. Then, silica precursor is added and reacted with stirring to prepare a silica nanoparticles dispersion.

(2) Preparation of Core-Shell Silica Particles with Single Mesopores

Silica nanoparticles dispersion is added to distilled water including ammonia solution, etc. in a reaction vessel, followed by stirring to prepare a solution A. Thereafter, a surfactant solution is stirred and added to the solution A, followed by stirring. Silica precursor is added with stirring, and heated to prepare a silica template.

### (3) Preparation of a Capsule-Type Silica-Titania Composite

The silica template prepared in the above step is dispersed in a mixed solution of an organic solvent and water. TiO₂ precursor can be prepared by adding titanium butoxide, etc. to a solvent such as ethylene glycol and then stirring it. The TiO₂ precursor is added to the dispersed silica template, followed by stirring, washing with an organic solvent and then drying it. Then, the resultant is subjected to thermal treatment to prepare a capsule type silica composite on which a metal or metal oxide layer is formed.

### (4) Preparation of a Porous Hollow Capsule

The prepared capsule type silica composite is dispersed in an organic solvent, and reacted appropriately, and then the silica template is removed to prepare a porous hollow capsule.

Hereinafter, the present invention will be described in more detail through the examples and preparation examples according to the present invention, but the scope of the present invention is not limited to the examples presented below.

### EXAMPLES

### Example 1

### Preparation of an Implant Fixture with a Hybrid Surface

(1) SiO₂ beads were prepared with a diameter not less than 100 nm and not more than 1 μm. SiO₂ nanocapsules were prepared according to the procedure below.

### 1) Preparation of Silica Nanoparticles Dispersion

Silica nanoparticles with various sizes were prepared by using the Stober method. In the sol-gel reaction, the type of solvent, amount of catalyst, amount of water, and the amount of tetraethylorthosilicate (TEOS), which is a precursor, etc. affect the size of particles. A more specific preparation procedure is as follows:

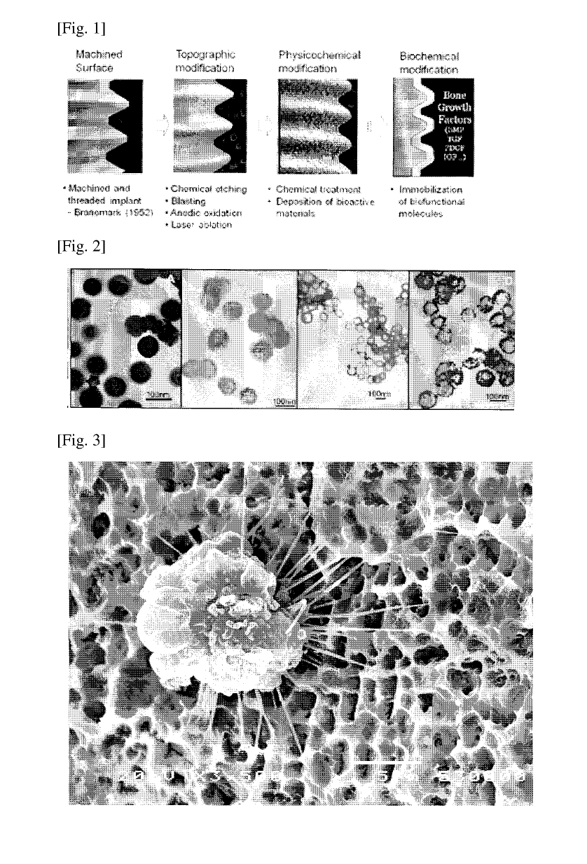

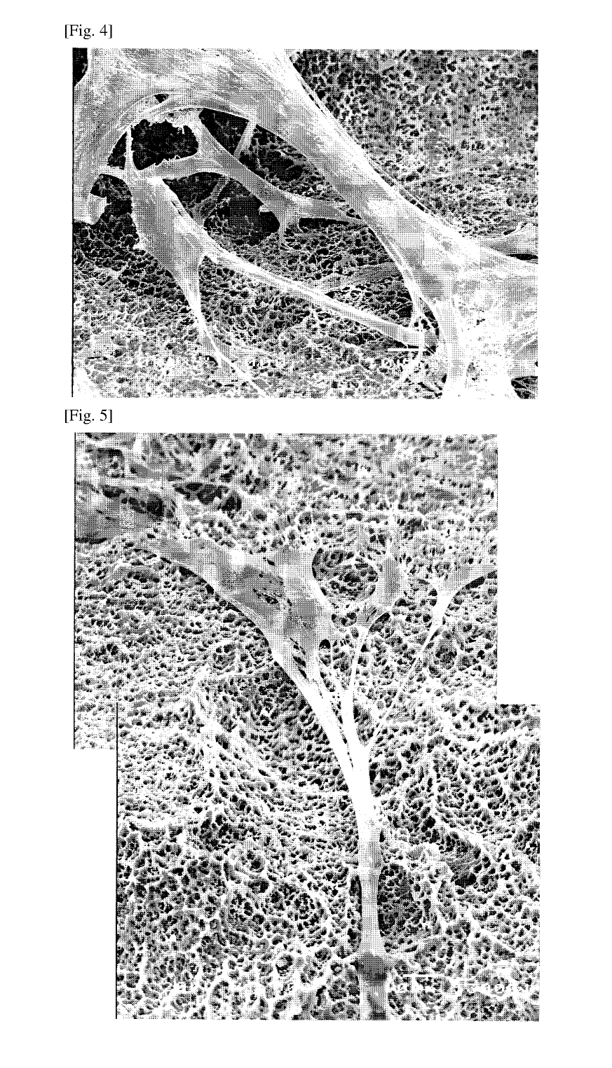

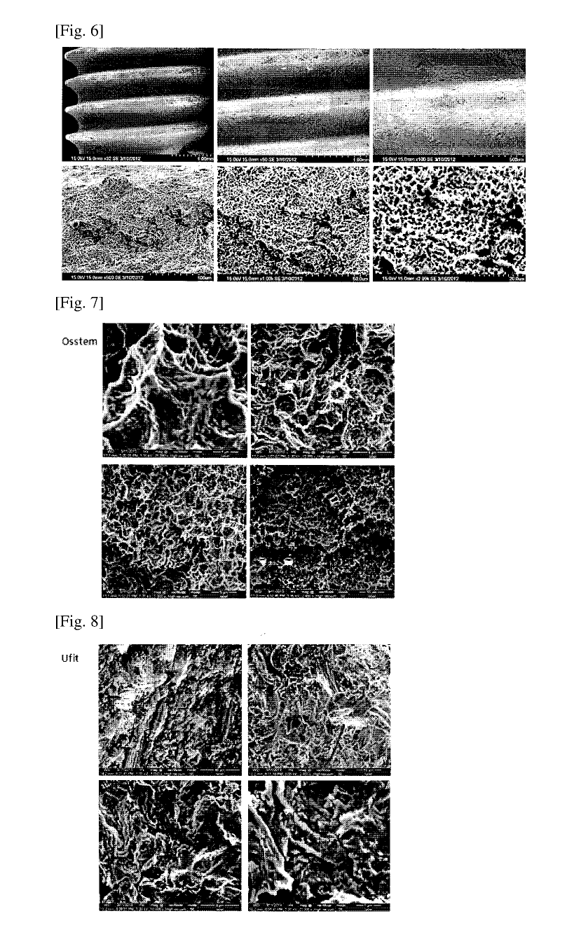

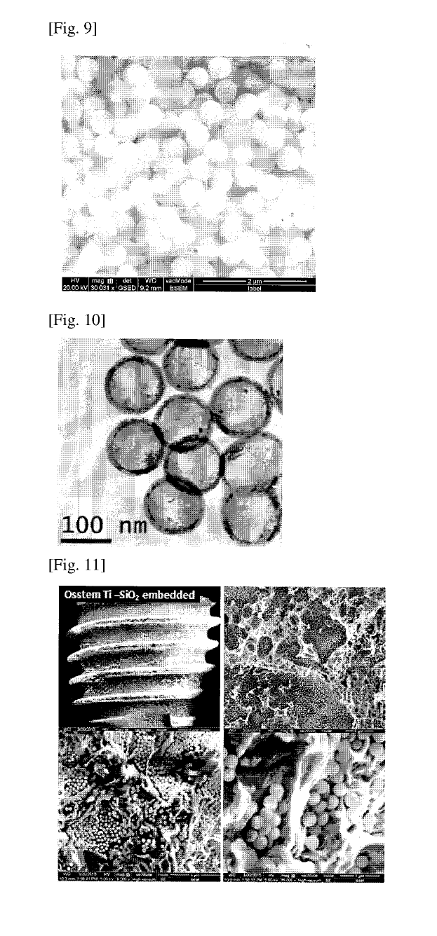

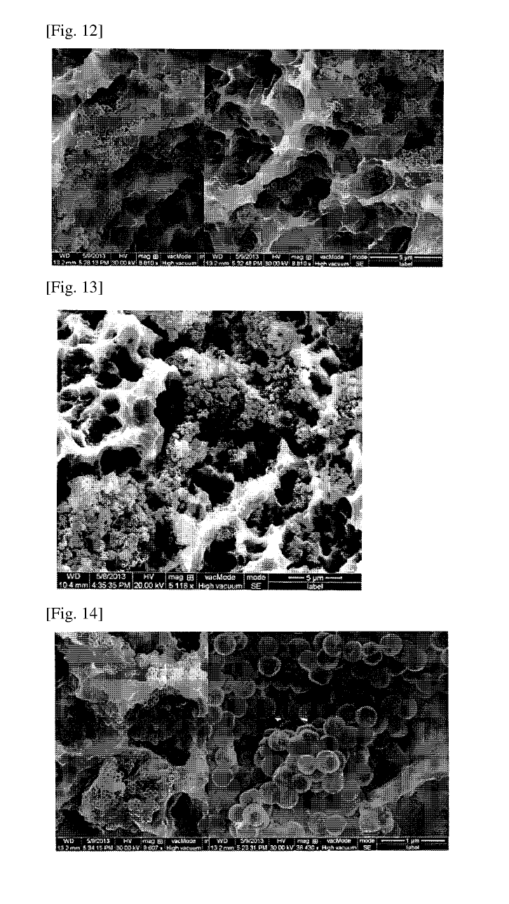

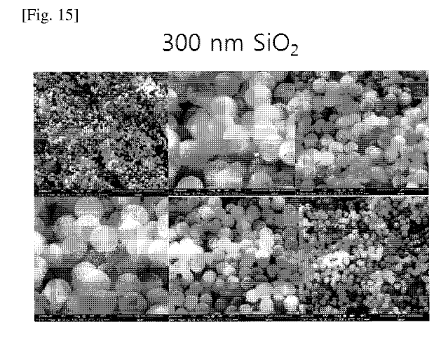

1,000 mL of ethanol and 10 mL of deionized water were put into a reaction vessel and then 1 mol of 28 wt % ammonia solution as a catalyst was added, followed by stirring at room temperature for 1 hour. Then, 0.14 mol of TEOS as a silica precursor was added and reacted with stirring at room temperature for 3 hours to prepare a silica nanoparticles dispersion. The resultant silica particles were about 500 nm in diameter (see FIG. 1).

2) Preparation of Core-Shell Silica Particles with Single Mesopores

10 mL of the silica nanoparticles dispersion prepared in step 1) was added to 20 mL of distilled water including ammonia solution (28 wt %, 0.1 mL) in a reaction vessel, followed by stirring for 30 minutes to prepare a solution A. 6.24 mL of a surfactant solution consisting of cetyltrimethylammonium bromide:1,3,5-trimethylbenzene:decane:distilled water:ethanol at the molar ratio of 1:1:1:113.99:17.77 was stirred at room temperature for 30 minutes and then added to the solution A, followed by stirring at room temperature for 30 minutes. Then, 0.43 mL of TEOS was added with stirring, followed by stirring for 10 minutes. After the stirring, the resultant was subjected to hydrothermal reaction in an oven set at 70° C. for 15 hours. The resultant sample was recovered by using a centrifuge, followed by drying at 70° C., gradual heating from room temperature up to 500° C. by using a tube furnace with oxygen blowing for 1 hour and 40 minutes, allowing it to stand at 500° C. for 5 hours, and then cooling it down back to room temperature to remove organic matter,

### 3) Preparation of a Capsule-Type Silica-Titania Composite

0.1 g of the silica template prepared in the step 2) was put into a mixed solution of 50 mL of acetone and 0.1 mL of deionized water and then dispersed by using an ultrasonic machine. TiO₂ precursor was prepared by adding 0.4 mL of titanium butoxide to 60 mL of ethylene glycol and stirring them for 12 hours. 10 mL of the TiO₂ precursor was added to the dispersed silica template, followed by stirring for 3 hours, washing with ethanol and then drying it at 70° C. for 12 hours. Then, the resultant was subjected to thermal treatment at 450° C. for 5 hours by using a tube furnace with flowing oxygen to prepare a capsule type silica composite on which a metal or metal oxide layer is formed. The thickness of the resultant oxide layer was 25 nm.

### 4) Preparation of a Porous Hollow Capsule

0.1 g of the resultant capsule type silica composite was dispersed in 3 mL of ethanol, and the dispersion was put into 5 mL of NaOH aqueous solution. The resultant was reacted in a reaction oven set at 70° C. for 3 to 5 hours to remove the silica template and thereby to prepare a porous hollow capsule. The resultant porous hollow capsule was separated by means of centrifugation, washed with ethanol, and dried at 70° C. for 12 hours.

(2) TiO₂, HA or TCP was coated onto SiO₂ bead templates using SiO₂ beads having the respective diameters as a template, and the SiO₂ beads template was removed (dissolved) to prepare TiO₂, HA or TCP hollow capsule. In order to prepare a capsule type silica-titania composite, the silica template prepared in the above step was put into a mixed solution of an organic solvent and water and then dispersed. Titania precursor was added to a mixed solution of an organic solvent such as ethylene glycol and water and then stirred to disperse the precursor uniformly into the solution. Here, when the precursor solution is added dropwise to the dispersed silica template, followed by vigorous stirring to proceed with reaction, coating starts with deposition of the precursor on the surface of the silica template through hydrolysis. The coating thickness is determined by the concentration and drop time of the precursor solution. After addition and stirring for a sufficient time of 30 minutes or more is completed, the coated capsule type silica-titania composite was centrifuged by using a centrifuge. Impurities adhered to these particles were washed with an organic solvent and air drying was performed. When thermal drying at 60° C. or higher is performed, a pure capsule type silica composite from which solvent and impurities have been completely removed can be prepared. Thereafter, a porous hollow capsule was prepared as follows: The prepared capsule type silica composite was uniformly dispersed in a solvent in which water and an organic solvent is mixed, and silica and strong base were appropriately diluted in an aqueous solution, followed by heating and stirring for 30 minutes or more. During this process, the silica template in the core reacts with the base ion of the aqueous solution which penetrated thereinto to release silica gradually, resulting in the preparation of a porous hollow capsule from which the core has been removed.

(3) The fixture surface of an implant (Osstem Implant Co., Ltd., RBM type, SLA type or Laser type) was sonicated for 5 to 10 minutes and then washed with alcohol. After the washing was completed, it was treated with silane and dried.

(4) The silica nanocapsule and hollow nanocapsule prepared through the steps (1) and (2) were put into the implant fixture prepared through the step (3), and then attached thereto by dipping and stirring or centrifugation. Thereafter, sonication was carried out to detach and remove an excess remnant of nanocapsules and hollow nanocapsules or those bonded with weak bonds. Thereby, an implant with a first hybrid surface, that is, an implant with a nano- and micro-scaled hybrid surface was prepared.

(5) It was shown that the hollow capsule prepared through the step (2) was not a closed capsule, but a spherical capsule with irregular pores of various sizes having a diameter 1/100 to 1/10 that of the capsule.

(6) The hollow nanocapsule attached to an implant with the first hybrid surface prepared through the step (4) exhibited pores not less than 20 nm and not more than 100 nm. Functional factors, such as peptide involved in promotion of osteogenesis, factors enhancing acidity, e.g., citric acid, ascorbic acid, medicines such as antibiotics, antibacterials, and anti-inflammatory drugs may be loaded within the hollow nanocapsule by a method(s) of dipping, centrifugation, sonication. Thereby, it is possible to prepare an implant with a hybrid surface having a drug delivery system (DDS) allowing a sustained release of functional factors (an implant with a second hybrid surface, that is, an implant with a hybrid surface having a DDS function).

### INDUSTRIAL APPLICABILITY

The implant according to the present invention induces rapid settling down of cells involved in osteogenesis and thereby inducing rapid wound healing and regeneration of solid bony tissue, and can also serve as a drug delivery system by virtue of the nanocapsules loaded therein. Therefore, the implant according to the present invention is very useful industrially.

1. A method for manufacturing an implant with a hybrid surface, comprising the steps of:
(a) treating the surface of an implant fixture with sonication, and washing it with a solvent;
(b1) preparing a SiO₂ bead, and preparing a nano-sized capsule by using it as a template, wherein a biocompatible material is coated on the SiO₂ template and then the SiO₂ bead is removed to prepare a hollow capsule, or (b2) preparing a bead with a biocompatible material; and
(c) attaching the hollow capsule prepared in step (b1) or the bead prepared in step (b2) to the implant fixture prepared in step (a) by dipping and stirring or centrifugation to prepare a first hybrid surface implant.

2. The method for manufacturing an implant with a hybrid surface according to claim 1, characterized in that the method further comprises, in step (b1), the step of loading a functional factor into the hollow capsule.

3. The method for manufacturing an implant with a hybrid surface according to claim 1, characterized in that the biocompatible material in step (b1) or step (b2) is selected from the group consisting of a titanium oxide selected from the group consisting of TiO₂, Ti₃O, Ti₂O, Ti₃O₂, TiO, Ti₂O₃, Ti₃O₅ and titanium butoxide; tricalcium phosphate, calcium phosphate; apatite selected from the group consisting of hydroxyapatite, hydroxyapatite substituted with silicon and magnesium; calcium sulfate; zirconium dioxide; silicon dioxide; and combinations thereof.

4. The method for manufacturing an implant with a hybrid surface according to claim 1, characterized in that the biocompatible material in step (b1) or step (b2) is one or more selected from the group consisting of TiO₂, hydroxyapatite and tricalcium phosphate.

5. The method for manufacturing an implant with a hybrid surface according to claim 1, characterized in that, in step (a), the diameter of the SiO₂ bead is not less than 500 nm and not more than 1 μm.

6. The method for manufacturing an implant with a hybrid surface according to claim 2, characterized in that the functional factor is selected from the group consisting of factors exhibiting the functions of promoting osteogenesis and enhancing antibacterial activity, anti-inflammatory activity and acidity, growth hormones, cell differentiation inducers, osteoblastic factors, and angiogenic factors.

7. The method for manufacturing an implant with a hybrid surface according to claim 2, characterized in that the functional factor is loaded into the hollow capsule by using one or more method selected from the group consisting of dipping, centrifugation and sonication.

8. An implant with a hybrid surface manufactured according to the method of claim 1.

9. The implant with a hybrid surface according to claim 8, characterized in that the implant is a dental implant.

10. An implant with a hybrid surface manufactured according to the method of claim 2.

11. An implant with a hybrid surface manufactured according to the method of claim 3

12. An implant with a hybrid surface manufactured according to the method of claim 4.

13. An implant with a hybrid surface manufactured according to the method of claim 5.

14. An implant with a hybrid surface manufactured according to the method of claim 6.

15. An implant with a hybrid surface manufactured according to the method of claim 7.
